(19) United States
(12) Patent Application Publication
(10) Pub. No.: US 2012/0122239 A1
(43) Pub. Date: May 17, 2012

(54) HUMAN NT-PRO B-TYPE NATRIURETIC PEPTIDE ASSAY HAVING REDUCED CROSS-REACTIVITY WITH OTHER PEPTIDE FORMS

(75) Inventors: John George Konrath, Lake Villa, IL (US); Jeffrey Allen Moore, Gurnee, IL (US)

(73) Assignee: ABBOTT LABORATORIES, Abbott Park, IL (US)

(21) Appl. No.: 13/344,183

(22) Filed: Jan. 5, 2012

Related U.S. Application Data
(63) Continuation of application No. 12/465,572, filed on May 13, 2009, now abandoned.

(51) Int. Cl. G01N 33/74

(52) U.S. Cl. 436/501

(57) ABSTRACT

The present disclosure relates to assays for detecting and/or quantifying the amount of human NT-pro B-type natriuretic peptide or human NT-pro B-type natriuretic peptide fragment in a test sample.

### RELATED APPLICATION INFORMATION

This application is a continuation of U.S. Ser. No. 12/465,572 filed on May 13, 2009, the contents of which are herein incorporated by reference.

### TECHNICAL FIELD

The present disclosure relates to assays for detecting and/or quantifying the amount of human NT-pro B-type natriuretic peptide or human NT-pro B-type natriuretic peptide fragment in a test sample. Specifically, the assays of the present disclosure exhibit less than about one percent (1.0%) cross-reactivity with any human pro B-type natriuretic peptide present or contained in a test sample.

### BACKGROUND

Atrial natriuretic peptide (hereinafter “ANP”), B-type natriuretic peptide (hereinafter “BNP”), C-type natriuretic peptide (hereinafter “CNP”) and Dendroaspis natriuretic peptide (hereinafter “DNP”) are each members of a family of hormones known as “natriuretic peptides”. ANP and BNP share a wide spectrum of biological properties and belong to the cardiac natriuretic system. Both ANP and BNP are of myocardial cell origin while CNP is of endothelial cell origin. DNP was isolated from the venom of the green mamba snake and possesses structural similarity to ANP, BNP and CNP.

ANP is secreted by the heart in the atria. ANP has a 17 amino acid ring closed by a disulfide bond between two cysteine residues. Eleven of the seventeen amino acids in the ring are conserved across ANP, BNP, CNP and DNP. In addition to the 17 amino acid ring structure, ANP has an amino-terminal tail of 6 amino acids and a carboxy-terminal tail of 5 amino acids. ANP is produced as a 126 amino acid pro-ANP form that is the major storage form of ANP. After proteolytic cleavage between amino acids 98 and 99, the mature 28 amino acid peptide ANP is found in coronary sinus plasma (See Yandle, J. Internal Med., 235:561-576 (1994)).

BNP received its name because it was first isolated from porcine brain, thus, initially, “BNP” stood for “brain natriuretic peptide”. However, because BNP was found to belong to the cardiac natriuretic system, the word “brain” was changed to “B-type”. Therefore, “BNP” now refers to “B-type natriuretic peptide”. In humans, BNP is secreted by the heart through the coronary sinus, predominantly from the cardiac ventricles. The pre-pro peptide precursor of human BNP (hereinafter “human pre-proBNP”) is 134 amino acids in length (SEQ ID NO:1) and comprises a short signal peptide, which is enzymatically cleaved off to release the human pro peptide of BNP (hereinafter “human proBNP”) which is 108 amino acids in length (SEQ ID NO:2). Human proBNP is further cleaved into an N-terminal pro peptide of human BNP (hereinafter “human NT-proBNP”) which is 76 amino acids in length (SEQ ID NO:3) and the active hormone, human BNP (hereinafter “hBNP” or “hBNP-32”), which is 32 amino acids in length (SEQ ID NO:4). It has been suggested that each of human NT pro-BNP, hBNP-32, and human proBNP—can circulate in human plasma (See, Tateyama et al., Biochem. Biophys. Res. Commun 185: 760-7 (1992); Hunt et al., Biochem. Biophys. Res. Commun 214: 1175-83 (1995)).

CNP was first found in the brain, however, most of it originates in endothelial and renal cells. It is widely distributed in the vasculature, brain, bone and endothelium. Little if any CNP is present in the heart. Pro-CNP is a 103 amino acid peptide that is processed into either CNP-53 (amino acids 51 to 103) or CNP-22 (amino acids 82 to 103) that are the active peptides. Like ANP, CNP has a 17 amino acid ring closed by a disulfide bond between cysteine residues. In addition to this 17 amino acid ring structure, CNP-22 has an amino-terminal tail of 5 amino acids and contains no carboxy-terminal tail. CNP-53 is identical to CNP-22 except for a 31 amino acid extension at the amino terminal end.

As mentioned previously, DNP was isolated from the venom of the green mamba snake. The mature form of DNP is made up of 38 amino acids. DNP-like immunoreactivity (DNP-LI) has been reported in human plasma and the plasma concentration of DNP-LI has been found to be elevated in patients with congestive heart failure (See, Cataliotti, et al., Mayo Clin. Proc., 76:111-1119 (2001)). Additionally, it is also known that the infusion of synthetic DNP results in marked natriuresis and diuresis in association with increased plasma and urinary cyclic guanosine monophosphate. Id.

In humans, heart disease can stimulate the secretion of ANP and BNP. In fact, the secretion of ANP and BNP in humans typically reflects a change in cardiac function. Specifically, the secretion of ANP is typically accelerated when the atrium undergoes a load, while the biosynthesis and secretion of BNP is stimulated when the ventricle undergoes a load. Thereupon, both ANP and BNP are useful as indicators in the diagnosis of heart disease. However, despite this and over time, BNP has become recognized as a useful indicator in the diagnosis of heart disease, more so than ANP. For example, the blood concentration of BNP is only ⅙ of ANP in a normal subject but it becomes higher than ANP in patients of heart failure. Moreover, the blood concentration of BNP increases in the case of heart failure like ANP, and the plasma concentration of BNP often exceeds that of ANP, thus reflecting more accurately the severity of heart dysfunction. Moreover, BNP level in patients of heart failure sometimes increases to several tens times to several hundreds times of that of healthy normal subjects.
It is known that human proBNP, human NT-proBNP and hBNP can circulate and may be detected in test samples of patients suffering from cardiovascular disease, particularly heart failure. Both hBNP and human NT-proBNP are frequently used as markers to detect heart failure and to assess risk thereof in patients. However, the actual amount of each of the individual forms of BNP (i.e. human proBNP, human NT-proBNP and human BNP) that circulate is unclear due to the cross-reactivities of current commercial assays for these various forms (See, Liang F., et al., J. American College of Cardiology, 49(10):1071-1078 (2007)).
Additionally, it is known that human proBNP and human NT-proBNP can be glycosylated (See, Schellenberger, U. et al., Archives of Biochemistry and Biophysics, 451:160-166 (2006)), and these glycosylated forms have been isolated from human samples (See, Hammerer-Lercher A., et al., Clinical Chemistry, 54(5):858-865 (2008) and Seferian, K. et al., Clinical Chemistry, 54(5):866-873 (2008)). There are seven sites of possible glycosylation confined to a 36-amino acid region within the N terminal portion of the peptide (from amino acid 36 through 71). Antibodies generated to this region may or may not bind to samples containing analyte human proBNP or NT-proBNP, depending on: 1) the immunogen used to raise the antibody; and 2) whether or not the analyte is glycosylated. Optional assays for human proBNP and NT-proBNP should use antibodies that avoid these regions. A number of high-affinity monoclonal antibodies that are specific for human NT-proBNP and human proBNP are known in the art. For example, HyTest News, dated June 2005, describes a number of such high affinity monoclonal antibodies. An example of antibodies disclosed are shown in the below Table A.

TABLE A

| | Cat. | | | | |
|---|---|---|---|---|---|
| Mab | # | Specificity | Subclass | Epitope | Application |
| 15F11 | 4NT1 | Human NT- | IgG2b | a.a.r. 13-24 | EIA, |
| | | proBNP, | | | Sandwich |
| | | proBNP | | | Immunoassay, |
| | | | | | WB |
| 24E11 | 4NT1 | Human NT- | IgG2a | a.a.r. 67-76 | EIA, |
| | | proBNP, | | | Sandwich |
| | | proBNP | | | Immunoassay, |
| | | | | | WB |
| 15C4 | 4NT1 | Human NT- | IgG2b | a.a.r. 63-71 | EIA, |
| | | proBNP, | | | Sandwich |
| | | proBNP | | | Immunoassay, |
| | | | | | WB |
| 29D12 | 4NT1 | Human NT- | IgG2a | a.a.r. 5-12 | EIA, |
| | | proBNP, | | | Sandwich |
| | | proBNP | | | Immunoassay, |
| | | | | | WB |
| 13G12 | 4NT1 | Human NT- | IgG2a | a.a.r. 13-20 | EIA, |
| | | proBNP, | | | Sandwich |
| | | proBNP | | | Immunoassay, |
| | | | | | WB |
| 18H5 | 4NT1 | Human NT- | IgG1 | a.a.r. 13-20 | EIA, |
| | | proBNP, | | | Sandwich |
| | | proBNP | | | Immunoassay, |
| | | | | | WB |

The HyTest News, dated June 2005 describes human NT-proBNP quantitative sandwich immunoassays and teaches that that monoclonal antibody pairs (capture-detection) 15F11-24E11, 15C4-29D12, 15C4-13G12 and 15C4-18H5 demonstrate high sensitivity in antigen recognition (10-15 pg/ml) and good kinetics. The HyTest News is completely silent on the sensitivity and cross-reactivity of said monoclonal antibody pairs with any human proBNP that might be in a test sample. Moreover, human proBNP cross reactivity with human NT-proBNP kits has been observed in the literature (See, Luckenbill, K., et al., Clin. Chem., 54:619-621 (2008)).
In view thereof, there is a need in the art for new assays for quantifying the amount of human NT-proBNP, particularly assays having reduced cross-reactivity with other forms of the peptide, and especially as any clinical significance of variance in their individual circulating concentrations (e.g., vis-à-vis other forms) becomes understood. The present disclosure seeks to provide new assays and methods. The present disclosure also seeks to provide a kit for use in such assays and methods. The methods and kit can be used in qualitative or quantitative assays for human NT-proBNP, including assays carried out to assess the severity of cardiovascular disease, monitor progression of cardiovascular disease, or assess risk of progression of cardiovascular disease. These and other objects and advantages, as well as other additional features, will become apparent from the detailed description provided herein.

### SUMMARY

In one embodiment, the present invention relates to an immunoassay for quantifying the amount of human NT-pro B-type natriuretic peptide (“human NT-proBNP”) in a test sample being tested for or suspected of containing human NT-proBNP, the immunoassay having reduced cross-reactivity with any human pro B-type natriuretic peptide (“human proBNP”). The immunoassay can comprise the steps of:

(a) contacting at least one capture antibody that binds to human NT-proBNP and that has been immobilized onto a solid phase to produce an immobilized antibody with said test sample to form a first mixture comprising an at least one capture antibody-human NT-proBNP complex, wherein the at least one capture antibody comprises an antibody that binds to an epitope comprising or consisting of amino acid residues 13-24 of SEQ ID NO:3;

(b) contacting said first mixture comprising the at least one capture antibody-human NT-proBNP complex with at least one detection antibody that binds to human NT-proBNP and that has been conjugated to a detectable label to form a second mixture comprising at least one capture antibody-human NT-proBNP-at least one detection antibody complex, wherein the at least one detection antibody comprises an antibody that binds to an epitope comprising or consisting of amino acid residues 63-71 of SEQ ID NO:3 or amino acid residues 67-76 of SEQ ID NO:3; and

(c) determining the amount of the at least one capture antibody-human NT-proBNP-at least one detection antibody complex formed in step (b) by detecting the detectable label as a measure of the amount of human NT-proBNP contained in the test sample,

wherein the at least one capture antibody and the at least one detection antibody, when used together, exhibit a cross-reactivity of less than about 1.0% with any human proBNP present in the test sample.

An example of an antibody that binds to an epitope comprising or consisting of amino acids residues 13-24 of SEQ ID NO:3 for use in the above immunoassay is antibody 15F11.

An example of an antibody that binds to an epitope comprising or consisting of amino acid residues 63-71 of SEQ ID NO:3 for use in the above immunoassay is antibody 15C4.

An example of an antibody that binds to an epitope comprising or consisting of amino acid residues 67-76 of SEQ ID NO:3 for use in the above immunoassay is antibody 24E11.

In another embodiment, the present invention relates to an immunoassay for quantifying the amount of human NT-proBNP in a test sample being tested for or suspected of containing human NT-proBNP, the immunoassay having reduced cross-reactivity with any human proBNP. The immunoassay can comprise the steps of:

(a) contacting at least one capture antibody that binds to human NT-proBNP and that has been immobilized onto a solid phase to produce an immobilized antibody with said test sample to form a first mixture comprising an at least one capture antibody-human NT-proBNP complex, wherein the at least one capture antibody comprises antibody 15F11;

(b) contacting said first mixture comprising the at least one capture antibody-human NT-proBNP complex with at least one detection antibody that binds to human NT-proBNP and that has been conjugated to a detectable label to form a second mixture comprising at least one capture antibody-human NT-proBNP-at least one detection antibody complex, wherein the at least one detection antibody comprises antibody 24E11 or 15C4; and

(c) determining the amount of the at least one capture antibody-human NT-proBNP-at least one detection antibody complex formed in step (b) by detecting the detectable label as a measure of the amount of human NT-proBNP contained in the test sample,

wherein the at least one capture antibody and the at least one detection antibody, when used together, exhibit a cross-reactivity of less than about 1.0% with any human proBNP present in the test sample.

In the above immunoassay, the at least one detection antibody can be 24E11.

Optionally, in the above immunoassay, the at least one detection antibody can be 15C4.

In still yet another embodiment, the present invention relates to an immunoassay for quantifying the amount of human NT-proBNP in a test sample being tested for or suspected of containing human NT-proBNP, the immunoassay having reduced cross-reactivity with any human proBNP present in the test sample. The immunoassay comprises the steps of:

(a) contacting said test sample with at least one detection antibody that binds to human NT-proBNP and that has been conjugated to a detectable label to form a first mixture comprising an at least one human NT-proBNP-detection antibody complex, wherein the at least one detection antibody comprises an antibody that binds to an epitope comprising or consisting of amino acid residues 63-71 of SEQ ID NO:3 or amino acid residues 67-76 of SEQ ID NO:3;

(b) contacting said first mixture comprising said at least one human NT-proBNP-detection antibody complex with at least one capture antibody that binds to human NT-proBNP and that has been immobilized on to a solid phase to produce an immobilized antibody to form a second mixture comprising an at least one capture antibody-human NT-proBNP-at least one detection antibody complex, wherein said at least one capture antibody comprises an antibody that binds to an epitope comprising or consisting of amino acid residues 13-24 of SEQ ID NO:3; and

(c) determining the amount of the at least one capture antibody-human NT-proBNP-at least one detection antibody complex formed in step (b) by detecting the detectable label as a measure of the amount of human NT-proBNP contained in the test sample,

wherein the at least one capture antibody and the at least one detection antibody, when used together, exhibit a cross-reactivity of less than about 1.0% with any human proBNP present in the test sample.

An example of an antibody that binds to an epitope comprising or consisting of amino acid residues 13-24 of SEQ ID NO:3 for use in the above immunoassay is antibody 15F11.

An example of an antibody that binds to an epitope comprising or consisting of amino acid residues 63-71 of SEQ ID NO:3 for use in the above immunoassay is antibody 15C4.

An example of an antibody that binds to an epitope comprising or consisting of amino acid residues 67-76 of SEQ ID NO:3 for use in the above immunoassay is antibody 24E11.

In still yet another embodiment, the present invention relates to an immunoassay for quantifying the amount of human NT-proBNP in a test sample being tested for or suspected of containing human NT-proBNP, the immunoassay having reduced cross-reactivity with any human proBNP present in the test sample. The immunoassay comprises the steps of:

(a) contacting said test sample with at least one detection antibody that binds to human NT-proBNP and that has been conjugated to a detectable label to form a first mixture comprising an at least one human NT-proBNP-detection antibody complex, wherein the at least one detection antibody comprises antibody 24E11 or 15C4;

(b) contacting said first mixture comprising said at least one human NT-proBNP-detection antibody complex with at least one capture antibody that binds to human NT-proBNP and that has been immobilized on to a solid phase to produce an immobilized antibody to form a second mixture comprising an at least one capture antibody-human NT-proBNP-at least one detection antibody complex, wherein said at least one capture antibody comprises antibody 15F11; and

(c) determining the amount of the at least one capture antibody-human NT-proBNP-at least one detection antibody complex formed in step (b) by detecting the detectable label as a measure of the amount of human NT-proBNP contained in the test sample,

wherein the at least one capture antibody and the at least one detection antibody, when used together, exhibit a cross-reactivity of less than about 1.0% with any human proBNP present in the test sample.

In the above immunoassay, the at least one detection antibody can be 24E11.

Optionally, in the above immunoassay, the at least one detection antibody can be 15C4.

In still yet another embodiment, the present invention relates to an immunoassay for quantifying the amount of human NT-proBNP in a test sample being tested for or suspected of containing human NT-proBNP, the immunoassay having reduced cross-reactivity with any human proBNP present in the test sample. The immunoassay comprises the steps of:

(a) contacting a test sample with at least one capture antibody that binds to human NT-proBNP and that has been immobilized onto a solid phase to produce an immobilized antibody and with at least one detection antibody that binds to human NT-proBNP and that has been conjugated to a detectable label to form an at least one capture antibody-human NT-proBNP-at least one detection antibody complex, wherein the at least one capture antibody comprises an antibody that binds to an epitope comprising or consisting of amino acid residues 13-24 of SEQ ID NO:3 and the at least one detection antibody comprises an antibody that binds to an epitope comprising or consisting of amino acid residues 63-71 of SEQ ID NO:3 or amino acid residues 67-76 of SEQ ID NO:3; and

(b) determining the amount of the at least one capture antibody-human NT-proBNP-at least one detection antibody complex formed in step (a) by detecting the detectable label as a measure of the amount of human NT-proBNP contained in the test sample,

wherein the at least one capture antibody and the at least one second antibody conjugated to the detectable label, when used together, exhibit a cross-reactivity of less than about 1.0% with any human proBNP present in the test sample.

An example of an antibody that binds to an epitope comprising or consisting of amino acid residues 13-24 of SEQ ID NO:3 for use in the above immunoassay is antibody 15F11.

An example of an antibody that binds to an epitope comprising or consisting of amino acids 63-71 of SEQ ID NO:3 for use in the above immunoassay is antibody 15C4.

An example of an antibody that binds to an epitope comprising or consisting of amino acid residues 67-76 of SEQ ID NO:3 for use in the above immunoassay is antibody 24E11.

In still yet another embodiment, the present invention relates to an immunoassay for quantifying the amount of human NT-proBNP in a test sample being tested for or suspected of containing human NT-proBNP, the immunoassay having reduced cross-reactivity with any human proBNP present in the test sample. The immunoassay comprises the steps of:

(a) contacting a test sample with at least one capture antibody that binds to human NT-proBNP and that has been immobilized onto a solid phase to produce an immobilized antibody and with at least one detection antibody that binds to human NT-proBNP and that has been conjugated to a detectable label to form an at least one capture antibody-human NT-proBNP-at least one detection antibody complex, wherein the at least one capture antibody comprises antibody 15F11 and the at least one detection antibody is antibody 24E11 or 15C4; and

(b) determining the amount of the at least one capture antibody-human NT-proBNP-at least one detection antibody complex formed in step (a) by detecting the detectable label as a measure of the amount of human NT-proBNP contained in the test sample,

wherein the at least one capture antibody and the at least one second antibody conjugated to the detectable label, when used together, exhibit a cross-reactivity of less than about 1.0% with any human proBNP present in the test sample.

In the above immunoassay, the at least one detection antibody can be 24E11.

Optionally, in the above immunoassay, the at least one detection antibody can be 15C4.

In still yet another embodiment, the present invention relates to an immunodiagnostic reagent comprising at least one capture antibody and at least one detection antibody specific for human NT-proBNP, and that, when used together, exhibit a cross-reactivity of less than about 1.0% with any human proBNP.
In the above described immunodiagnostic reagent, the at least one capture antibody comprises an antibody that binds to an epitope comprising or consisting of amino acid residues 13-24 of SEQ ID NO:3 and the at least one detection antibody comprises an antibody that binds to an epitope comprising or consisting of amino acid residues 63-71 of SEQ ID NO:3 or amino acid residues 67-76 of SEQ ID NO:3.
An example of a capture antibody that binds to an epitope comprising or consisting of amino acid residues 13-24 of SEQ ID NO:3 is antibody 15F11.

An example of a detection antibody that antibody that binds to an epitope comprising or consisting of amino acid residues 63-71 of SEQ ID NO:3 is antibody 15C4.

An example of a detection antibody that binds to an epitope comprising or consisting of amino acid residues 67-76 of SEQ ID NO:3 is antibody 24E11.

In still yet another embodiment, the present invention relates to a kit for the detection of human NT-proBNP in a test sample. The kit can comprise:
(a) instructions for conducting an assay of the test sample; and
(b) an immunodiagnostic reagent that comprises at least one capture antibody and at least one detection antibody specific for human NT-proBNP, and that, when used together, exhibit a cross-reactivity of less than about 1.0% with any human proBNP.
In the above kit, the at least one capture antibody comprises an antibody that binds to an epitope comprising or consisting of amino acid residues 13-24 of SEQ ID NO:3 and the at least one detection antibody comprises an antibody that binds to an epitope comprising or consisting of amino acid residues 63-71 of SEQ ID NO:3 or amino acid residues 67-76 of SEQ ID NO:3.
An example of a capture antibody that binds to an epitope comprising or consisting of amino acid residues 13-24 of SEQ ID NO:3 is antibody 15F11.

An example of a detection antibody that antibody that binds to an epitope comprising or consisting of amino acid residues 63-71 of SEQ ID NO:3 is antibody 15C4.

An example of a detection antibody that binds to an epitope comprising or consisting of amino acid residues 67-76 of SEQ ID NO:3 is antibody 24E11.

### DETAILED DESCRIPTION

The present disclosure relates to immunoassays for quantifying the amount of human NT-pro B-type natriuretic peptide (“human NT-proBNP”) present in a test sample being tested for or suspected of containing human NT-proBNP. Specifically, the immunoassays of the present disclosure exhibit reduced cross-reactivity with any human pro B-type natriuretic peptide (“human proBNP”) present in the test sample. In another embodiment, the present disclosure relates to immunoassays for quantifying the amount of human NT-proBNP in a test sample wherein the immunoassays exhibit reduced cross-reactivity with human proBNP. In still yet another embodiment, the present invention relates to an immunodiagnostic reagent comprising at least one capture antibody and at least one detection antibody specific for human NT-proBNP, and that, when used together, exhibit a cross-reactivity of less than about 1.0% with any human proBNP present in the test sample. In still yet another embodiment, the present invention relates to a kit for performing an immunoassay. Such kits can comprise instructions for conducting such an immunoassay and the above described immunodiagnostic reagent.

### A. DEFINITIONS

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

### a) Antibody

As used herein, the terms “antibody” and “antibodies” refer to monoclonal antibodies, multispecific antibodies, human antibodies, humanized antibodies (fully or partially humanized), animal antibodies (in one aspect, a bird (for example, a duck or goose), in another aspect, a shark or whale, in yet another aspect, a mammal, including a non-primate (for example, a cow, pig, camel, llama, horse, goat, rabbit, sheep, hamsters, guinea pig, cat, dog, rat, mouse, etc) and a non-human primate (for example, a monkey, such as a cynomologous monkey, a chimpanzee, etc)), recombinant antibodies, chimeric antibodies, single-chain Fvs (scFv), single chain antibodies, single domain antibodies, Fab fragments, F(ab′) fragments, Fab″ fragments, disulfide-linked Fvs (sdFv), and anti-idiotypic (anti-Id) antibodies (including, for example, anti-Id antibodies to antibodies of the present disclosure), and functionally active epitope-binding fragments of any of the above. In particular, antibodies include immunoglobulin molecules and immunologically active fragments of immunoglobulin molecules, namely, molecules that contain an antigen binding site. Immunoglobulin molecules can be of any type (for example, IgG, IgE, IgM, IgD, IgA and IgY), class (for example, IgG1, IgG2, IgG3, IgG4, IgA1 and IgA2) or subclass.

### b) 15C4

As used herein, the term “15C4” refers to an IgG2b monoclonal antibody that is available from HyTest (Turku, Finland) (Cat. #4NT1) that binds to amino acid residues 63-71 of SEQ ID NO:3.

### c) 15F11

As used herein, the term “15F11” refers to an IgG2b monoclonal antibody that is available from HyTest (Turku, Finland) (Cat. #4NT1) that binds to amino acid residues 13-24 of SEQ ID NO:3.

### d) 24E11

As used herein, the term “24E11” refers to an IgG2a monoclonal antibody that is available from HyTest (Turku, Finland) (Cat. #4NT1) that binds to amino acid residues 67-76 of SEQ ID NO:3.

### e) Epitope

As used herein, the term “epitope” or “epitopes” refers to sites or fragments of a polypeptide or protein having antigenic or immunogenic activity in a subject. An epitope having immunogenic activity is a site or fragment of a polypeptide or protein that elicits an antibody response in an animal. An epitope having antigenic activity is a site or fragment of a polypeptide or protein to which an antibody immunospecifically binds as determined by any method well-known to those skilled in the art, for example by immunoassays.

### f) Human Brain Natriuretic Peptide

As used herein, the terms “human brain natriuretic peptide”, “human BNP”, “hBNP”, “hBNP-32”, “hBNP peptide”, “hBNP polypeptide”, or “B-type natriuretic peptide” used interchangeably herein, refer to a 32 amino acid molecule having the amino acid sequence shown in SEQ ID NO:4. The amino acid sequence shown in SEQ ID NO:4 is represented by amino acid residues 77-108 of the 108 amino acid sequence of human proBNP (SEQ ID NO:2).

g) hBNP Fragment

As used herein, the terms “hBNP fragment” “hBNP-32 fragment”, “hBNP peptide fragment” or “human BNP fragment” as used interchangeably herein refers to a polypeptide that comprises at least six contiguous amino acid residues of SEQ ID NO:4. In one aspect, a hBNP fragment or hBNP peptide fragment refers to a peptide that comprises at least ten contiguous amino acid residues residues of SEQ ID NO:4; at least fifteen contiguous amino acid residues of amino acid residues of SEQ ID NO:4; at least 20 contiguous amino acid residues of SEQ ID NO:4; at least 25 contiguous amino acid residues of SEQ ID NO:4, or at least 30 contiguous amino acid residues of amino acids of SEQ ID NO:4. Examples of hBNP fragments or hBNP peptide fragments include, but are not limited to, amino acid sequences containing amino acids residues 1-31, 1-30, 1-29, 1-28, 1-27, 1-26, 1-25, 1-24, 1-23, 1-22, 1-21, 1-20, 1-19, 1-18, 1-17, 1-16, 1-15, 2-32, 2-31, 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 3-32, 3-31, 3-30, 3-29, 3-28, 3-27, 3-26, 3-25, 3-24, 3-23, 3-32, 3-21, 3-20, 3-19, 3-18, 3-17, 3-16, 3-15, 3-14, 3-13, 3-12, 3-11, 3-10, 3-9, 3-8, 4-32, 4-31, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 7-32, 7-31, 7-30, 7-29, 7-28, 7-27, 7-26, 7-25, 7-24, 7-23, 7-22, 7-21, 7-20, 7-19, 7-18, 7-17, 7-16, 7-15, 7-14, 7-13, 7-12, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 9-32, 9-31, 9-30, 9-29, 9-28, 9-27, 9-26, 9-25, 9-24, 9-23, 9-22, 9-21, 9-20, 9-19, 9-18, 9-17, 9-16, 9-15, 9-14, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 11-32, 11-31, 11-30, 11-29, 11-28, 11-27, 11-26, 11-25, 11-24, 11-23, 11-22, 11-21, 11-20, 11-19, 11-18, 11-17 or 11-16 of SEQ ID NO:4.

### h) Immunodiagnostic Reagent

As used herein, the term “immunodiagnostic reagent” refers to one or more antibodies that specifically bind to a region (e.g., epitope) of human NT-proBNP.

### i) Pre-Pro Peptide Precursor of Human BNP

As used herein, the term “pre-pro peptide precursor of human BNP” or “human pre-proBNP” refers to a 134 amino acid molecule having the amino acid sequence shown in SEQ ID NO:1.

### j) Human Pro B-Type Natriuretic Peptide

As used herein, the phrase “human pro B-type natriuretic peptide” or “human proBNP” refers to a 108 amino acid molecule having the amino acid sequence shown in SEQ ID NO:2. Human proBNP is derived from human pre-proBNP.

### k) Human N-Terminal-pro B-type Natriuretic Peptide

As used herein, the phrase “Human N-terminal-pro B-type natriuretic peptide” or “human NT-proBNP”, refers to a 76 amino acid molecule having the amino acid sequence shown in SEQ ID NO:3. Human NT-proBNP is derived from human proBNP (SEQ ID NO:2).

### l) Human N-Terminal-pro B-type Natriuretic Peptide Fragment

As used herein, the phrases “human N-terminal-pro B-type natriuretic peptide fragment” or “human NT-proBNP fragment” as used interchangeably herein refers to a polypeptide that comprises a fragment of a human NT-proBNP peptide. The fragment of a human NT-proBNP peptide contains a contiguous or nonlinear epitope of the human NT-proBNP peptide. The precise boundaries of such an epitope fragment can be confirmed using ordinary skill in the art. Specifically, the human NT-proBNP fragment of the present invention comprises at least two epitopes, specifically a first epitope comprising or consisting of amino acid residues 13-24 of SEQ ID NO:3 and a second epitope comprising or consisting of amino acid residues 63-71 or amino acid residues 67-76 of SEQ ID NO:3. Examples of human NT-proBNP fragment include, but are not limited to, amino acid sequences containing amino acid residues 1-75, 1-74, 1-73, 1-72, 1-71, 2-76, 2-75, 2-74, 2-73, 2-72, 2-71, 3-76, 3-75, 3-74, 3-73, 3-72, 3-71, 4-76, 4-75, 4-74, 4-73, 4-72, 4-71, 5-76, 5-75, 5-74, 5-73, 5-72, 5-71, 6-76, 6-75, 6-74, 6-73, 6-72, 6-71, 7-76, 7-75, 7-74, 7-73, 7-72, 7-71, 8-76, 8-75, 8-74, 8-73, 8-72, 8-71, 9-76, 9-75, 9-74, 9-73, 9-72, 9-71, 10-76, 10-75, 10-74, 10-73, 10-72, 10-71, 11-76, 11-75, 11-74, 11-73, 11-72, 11-71, 12-76, 12-75, 12-74, 12-73, 12-72, 12-71, 13-76, 13-75, 13-74, 13-73, 13-72, 13-71 of SEQ ID NO:3.

### m) Subject

As used herein, the terms “subject” and “patient” are used interchangeably, although a subject of the disclosure herein need not necessarily be undergoing or have undergone medical treatment at the time of the immunoassay. As used herein, the terms “subject” and “subjects” refer to an animal, in one aspect, a bird (for example, a duck or goose), in another aspect, a shark or whale, or in a further aspect, a mammal including, a non-primate (for example, a cow, pig, camel, llama, horse, goat, rabbit, sheep, hamsters, guinea pig, cat, dog, rat, and mouse) and a primate (for example, a monkey, such as a cynomologous monkey, chimpanzee, and a human). Preferably, the subject is a human.

### n) Test Sample

As used herein, the term “test sample” refers to a biological sample derived from tissues, serum, plasma, whole blood, lymph, CNS fluid, urine or other bodily fluids of a subject that is being tested for, and/or may be suspected of containing human NT-proBNP. The test sample can be prepared using routine techniques known to those skilled in the art.

### B. IMMUNOASSAYS AND IMMUNODIAGNOSTIC REAGENTS

As mentioned briefly herein, in one embodiment, the present disclosure relates to immunoassays for the qualitative detection and/or quantification of human NT-proBNP or human NT-proBNP fragment in a test sample. The immunoassays described herein exhibit reduced cross-reactivity with any human proBNP that may be contained in the test sample.

The immunoassays of the present disclosure can be conducted using any format known in the art, such as, but not limited to, a sandwich format.

In certain embodiments of the present disclosure, at least two antibodies are employed to separate and quantify human NT-proBNP or human NT-proBNP fragment in a test sample. More specifically, the at least two antibodies bind to certain epitopes of human NT-proBNP or human NT-proBNP fragment forming an immune complex which is referred to as a “sandwich”. Generally, in the immunoassays one or more antibodies can be used to capture the human NT-proBNP or human NT-proBNP fragment in the test sample (these antibodies are frequently referred to as a “capture” antibody or “capture” antibodies) and one or more antibodies can be used to bind a detectable (namely, quantifiable) label to the sandwich (these antibodies are frequently referred to as the “detection antibody,” “detection antibodies,” a “conjugate” or “conjugates”).

The inventors have discovered that excellent immunoassays, particularly, sandwich assays, can be performed using certain combinations of antibodies as the capture and detection antibodies. More specifically, the at least one capture antibody used in the present invention is an antibody that binds to an epitope comprising or consisting of amino acid residues 13-24 of SEQ ID NO:3. An example of an antibody that binds to an epitope comprising or consisting of amino acid residues 13-24 of SEQ ID NO:3 is antibody 15F11. The at least one detection antibody used in the present invention is an antibody that binds to an epitope comprising or consisting of amino acid residues 63-71 of SEQ ID NO:3 or amino acid residues 67-76 of SEQ ID NO:3. An example of an antibody that binds to an epitope comprising or consisting of amino acid residues 63-71 of SEQ ID NO:3 is antibody 15C4. An example of an antibody that binds to an epitope comprising or consisting of amino acid residues 67-76 of SEQ ID NO:3 is antibody 24E11.
Immunoassays performed as described herein that employ as at least one capture antibody that binds to an epitope comprising or consisting of amino acid residues 13-24 of SEQ ID NO:3 and at least one detection antibody that binds to an epitope comprising or consisting of amino acid residues 63-71 of SEQ ID NO:3 or amino acid residues 67-76 of SEQ ID NO:3, exhibit reduced cross-reactivity with any human proBNP that may be present in the test sample. Preferably, the immunoassay exhibits a cross-reactivity of less than about 1.0% with any human proBNP that may be present in the test sample. More preferably, the immunoassay exhibits a cross-reactivity of less than about 0.9%, less than about 0.8%, less than about 0.7%, less than about 0.6%, less than about 0.5%, less than about 0.4%, less than about 0.3%, less than about 0.2%, less than about 0.1% or less than about 0.001% with any human proBNP that may be present in the test sample.
In another embodiment of the disclosure, immunoassays performed as described herein that employ as at least one capture antibody that binds to an epitope comprising or consisting of amino acid residues 13-24 of SEQ ID NO:3 and at least one detection antibody that binds to an epitope comprising or consisting of amino acid residues 63-71 of SEQ ID NO:3 or amino acid residues 67-76 of SEQ ID NO:3, exhibit a reduced cross-reactivity with an human proBNP that may be present in the test sample. Preferably, the immunoassay exhibits a cross-reactivity of less than about 1.0% with any human proBNP that may be present in the test sample. More preferably, the immunoassay exhibits a cross-reactivity of less than about 0.9%, less than about 0.8%, less than about 0.7%, less than about 0.6%, less than about 0.5%, less than about 0.4%, less than about 0.3%, less than about 0.2%, less than about 0.1% or less than about 0.001% with any human proBNP that may be present in the test sample.

The test sample being tested for (e.g., suspected of containing) human NT-proBNP or a human NT-proBNP fragment can be contacted with at least one capture antibody (or antibodies) and at least one detection antibody (or antibodies) either simultaneously or sequentially and in any order. For example, the test sample can be first contacted with at least one capture antibody and then (sequentially) with at least one detection antibody. Alternatively, the test sample can be first contacted with at least one detection antibody and then (sequentially) with at least one capture antibody. In yet another alternative, the test sample can be contacted simultaneously with a capture antibody and a detection antibody. In the sandwich assay format, a test sample suspected of containing human NT-proBNP or human NT-proBNP fragment is first brought into contact with the at least one first capture antibody that binds to an epitope comprising or consisting of amino acid residues 13-24 of SEQ ID NO:3 under conditions which allow the formation of a first antibody-human NT-proBNP complex. If more than one capture antibody is used, a first multiple capture antibody-human NT-proBNP complex is formed. In a sandwich assay, the antibodies, preferably, the at least one capture antibody, are used in molar excess amounts of the maximum amount of human NT-proBNP or human NT-proBNP fragment expected in the test sample. For example, from about 5 μg/mL to about 1 mg/mL of antibody per mL of buffer (e.g., microparticle coating buffer) can be used.

Optionally, prior to contacting the test sample with the at least one capture antibody (e.g., the first capture antibody), the at least one capture antibody can be bound to a solid support which facilitates the separation of the first antibody-human NT-proBNP complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind human NT-proBNP or human NT-proBNP fragment. Alternatively, the antibody (or antibodies) can be bound with microparticles that have previously been coated with streptavidin (for example, using Power-Bind™-SA-MP streptavidin coated microparticles, available from Seradyn, Indianapolis, Ind.). Alternatively, the antibody (or antibodies) can be bound using microparticles that have been previously coated with anti-species specific monoclonal antibodies. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample being tested for and/or suspected of containing human NT-proBNP or an human NT-proBNP fragment is brought into contact with the at least one capture antibody (e.g., the first capture antibody), the mixture is incubated in order to allow for the formation of a first antibody (or multiple antibody)-human NT-proBNP complex. The incubation can be carried out at a pH of from about 4.5 to about 10.0, at a temperature of from about 2° C. to about 45° C., and for a period from at least about one (1) minute to about eighteen (18) hours, preferably from about 1 to 20 minutes, most preferably from about 2-4 minutes. The immunoassay described herein can be conducted in one step (meaning the test sample, at least one capture antibody and at least one detection antibody are all added sequentially or simultaneously to a reaction vessel) or in more than one step, such as two steps, three steps, etc.

After formation of the (first/multiple) capture antibody-human NT-proBNP complex, the complex is then contacted with at least one detection antibody (under conditions which allow for the formation of a (first/multiple) capture antibody-human NT-proBNP-second antibody detection complex). The at least one detection antibody can be the second, third, fourth, etc. antibodies used in the immunoassay. If the capture antibody-human NT-proBNP complex is contacted with more than one detection antibody, then a (first/multiple) capture antibody-human NT-proBNP-(multiple) detection antibody complex is formed. As with the capture antibody (e.g., the first capture antibody), when the at least second (and subsequent) detection antibody is brought into contact with the capture antibody-human NT-proBNP complex, a period of incubation under conditions similar to those described above is required for the formation of the (first/multiple) capture antibody-human NT-proBNP-(second/multiple) detection antibody complex. Preferably, at least one detection antibody contains a detectable label. The detectable label can be bound to the at least one detection antibody (e.g., the second detection antibody) prior to, simultaneously with or after the formation of the (first/multiple) capture antibody-human NT-proBNP-(second/multiple) detection antibody complex. Any detectable label known in the art can be used. For example, the detectable label can be a radioactive label, such as, 3H, 125I, 35S, 14C, 32P, 33P, an enzymatic label, such as horseradish peroxidase, alkaline phosphatase, glucose 6-phosphate dehydrogenase, etc., a chemiluminescent label, such as, acridinium esters, luminol, isoluminol, thioesters, sulfonamides, phenanthridinium esters, etc. a fluorescence label, such as, fluorescein (5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2nd ed., Springer Verlag, N.Y. (1997) and in Haugland, Handbook of Fluorescent Probes and Research Chemicals (1996), which is a combined handbook and catalogue published by Molecular Probes, Inc., Eugene, Oreg. In addition, more than one label can be used. For example, double conjugates can be used, each of which contain different labels. For example, one conjugate antibody can contain biotin and the second conjugate can be an anti-biotin antibody labeled with acridinium. Other variations would be easily recognized by one of ordinary skill in the art.

The detectable label can be bound to the antibodies either directly or through a coupling agent. An example of a coupling agent that can be used is EDAC (1-ethyl-3-(3-dimethylaminopropyl) carbodiimide, hydrochloride) that is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide active esters, otherwise known as CPSP-Acridinium Ester or N10-(3-sulfopropyl)-N-(3-sulfopropyl)-acridinium-9-carboxamide active ester, otherwise known as SPSP-Acridinium Ester.

The (first/multiple) capture antibody-human NT-proBNP-(second/multiple) detection antibody complex can be, but does not have to be, separated from the remainder of the test sample prior to quantification of the label. For example, if the at least one capture antibody (e.g., the first capture antibody) is bound to a solid support, such as a well or a bead, separation can be accomplished by removing the fluid (of the test sample) from contact with the solid support. Alternatively, if the at least first capture antibody is bound to a solid support it can be simultaneously contacted with the human NT-proBNP-containing sample and the at least one second detection antibody to form a first (multiple) antibody-human NT-proBNP-second (multiple) antibody complex, followed by removal of the fluid (test sample) from contact with the solid support. If the at least one first capture antibody is not bound to a solid support, then the (first/multiple) capture antibody-human NT-proBNP-(second/multiple) detection antibody complex does not have to be removed from the test sample for quantification of the amount of the label.

After formation of the labeled capture antibody-human NT-proBNP-detection antibody complex (e.g., the first capture antibody-human NT-proBNP-second detection antibody complex), the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. Once the amount of the label in the complex has been quantified, the concentration of human NT-proBNP or human NT-proBNP fragment in the test sample is determined by use of a standard curve that has been generated using serial dilutions of human NT-proBNP or human NT-proBNP fragment of known concentration. Other than using serial dilutions of human NT-proBNP or human NT-proBNP fragment, the standard curve can be generated gravimetrically, by mass spectroscopy and by other techniques known in the art.

In another embodiment, the present disclosure relates to immunodiagnostic reagents. Specifically, the at least one capture antibody (namely, an antibody that binds to an epitope comprising or consisting of amino acid residues 13-24 of SEQ ID NO:3) and at least one detection antibody (namely, an antibody that binds to an epitope comprising or consisting of amino acid residues 63-71 of SEQ ID NO:3 or amino acid residues 67-76 of SEQ ID NO:3) described herein can be used individually or in combination, as one or more immunodiagnostic reagents in one or more immunoassays, such as those described above. When the at least one capture antibody and at least one detection antibody described herein are used together in an immunoassay (such as those described previously herein), the immunoassay exhibits a cross-reactivity of less than about 1.0% with any human proBNP in a test sample. More specifically, the immunoassay exhibits less than about 0.9%, less than about 0.8%, less than about 0.7%, less than about 0.6%, less than about 0.5%, less than about 0.4%, less than about 0.3%, less than about 0.2%, less than about 0.1% or less than about 0.001% with any human proBNP that may be present in the test sample.

### C. ADAPTATIONS OF THE METHODS

The disclosure herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM®, IMx®, PRISM®, and Quantum™ II instruments, as well as other platforms. Moreover, the disclosure optionally is adaptable for the Abbott Laboratories commercial Point of Care (i-STAT™) electrochemical immunoassay system for performing sandwich immunoassays Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

### D. EXEMPLARY KITS

The present disclosure herein also can be adapted for use in a variety of kits for use on automated and semi-automated systems and platforms, e.g., commercially marketed by Abbott Laboratories (Abbott Park, Ill.) including, but not limited to, Abbott Laboratories' ARCHITECT®, AxSYM®, IMx®, PRISM®, and Quantum™ II instruments, Abbott Laboratories' commercial Point of Care (i-STAT™) electrochemical immunoassay system for performing sandwich immunoassays, as well as other platforms.
Such kits can comprise one or more of the immunodiagnostic reagents (e.g., the capture and detection antibodies) described herein. More specifically, if the kit is a kit for performing an immunoassay, the kit can optionally contain (1) at least one capture and detection antibody that bind to human NT-proBNP or human NT-proBNP fragment and together exhibit reduced cross-reactivity with human proBNP; and (2) one or more instructions for performing the immunoassay. The immunodiagnostic reagents of the present disclosure can be included in such a test kit as a capture antibody, as a detection antibody or both as a capture antibody and a detection antibody. For example, antibody 15F11 can be included in the kit as capture antibody and antibody 15C4 can be included in the kit as a detection antibody. Alternatively, antibody 15F11 can be included in the kit as a capture antibody and antibody 24E11 can be included in the kit as a detection antibody. Optionally, the kit can also contain at least one calibrator or control. Any calibrator or control can be included in the kit. Preferably, however, the calibrator or control is human NT-proBNP or human NT-proBNP fragment, especially SEQ ID NO: 3 described previously herein. Accordingly, the kits can comprise at least one calibrator, or at least one control, or a combination of at least one calibrator and at least one control.
Optionally the kits also can include quality control reagents (e.g., sensitivity panels, calibrators, and positive controls). Preparation of quality control reagents is well known in the art, and is described, e.g., on a variety of immunodiagnostic product insert sheets. Human NT-proBNP sensitivity panel members optionally can be prepared in varying amounts containing, e.g., known quantities of human NT-proBNP antigen ranging from “low” to “high”, e.g., by spiking known quantities of the human NT-proBNP antigen into an appropriate assay buffer (e.g., a phosphate buffer). These sensitivity panel members optionally are used to establish assay performance characteristics, and further optionally are useful indicators of the integrity of the immunoassay kit reagents, and the standardization of assays. The human NT-proBNP antigen also can be employed as calibrators.
The antibodies provided in the kit can incorporate a detectable label, such as a fluorophore, radioactive moiety, enzyme, biotin/avidin label, chromophore, chemiluminescent label, or the like, or the kit may include reagents for labeling the antibodies or reagents for detecting the antibodies (e.g., detection antibodies) and/or for labeling the antigens or reagents for detecting the antigen. The antibodies, calibrators and/or controls can be provided in separate containers or pre-dispensed into an appropriate assay format, for example, into microtiter plates.
In yet another embodiment, the kit can comprise, either alone, with instructions, or with other aspects of the kit and kit components, an immunodiagnostic agent that comprises one or more antibodies selected from the group consisting of 15F11, 15C4 and 24E11.
The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.
The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.
The kit further can optionally include instructions for use, which may be provided in paper form or in computer-readable form, such as a disc, CD, DVD or the like.
Now by way of example, and not of limitation, examples of the present disclosure shall now be given.

### Example 1

### Elimination of Detection of Human proBNP during Immunoassay for Human NT-proBNP

Materials, Methods and Results

In this example, an automated ARCHITECT® System (Abbott Laboratories, Abbott Park, Ill.) was used to perform an immunoassay that would quantitate human NT-proBNP in specimens that contained a range of concentrations of human NT proBNP (HyTest, Turku, Finland; Catalog No. 8NT1) in BNP Calibrator Diluent which contains 10 mM NaOAc, 10 mM DTPA, 2% BSA, 0.1% ProClin 300, 0.1% NaN3, pH 5.6 (Abbott Laboratories, Abbott Park, Ill.).

Dilutions of human NT proBNP (HyTest, Turku, Finland; Catalog No. 8NT1) containing human NT proBNP, (0.0 pM-577.4 pM) were tested. Testing was performed in reaction vessels (Abbott Laboratories, Abbott Park, Ill.) that are used for individual tests in the automated Abbott ARCHITECT® System. All the described steps took place in the ARCHITECT® instrument.
Dilutions of human proBNP (HyTest, Turku, Finland; Catalog No. 8PRO8) (0.0 pM-577.4 pM) and glycosylated human proBNP (HyTest, Turku, Finland; Catalog No. 8GOB2) (0.0 pM-577.4 pM) were simultaneously tested to determine the extent of cross reactivity. Testing was performed in reaction vessels (Abbott Laboratories, Abbott Park, Ill.) that are used for individual tests in the automated Abbott ARCHITECT® System. All the described steps took place in the ARCHITECT® instrument.

Specimens containing human NT-proBNP (HyTest, Turku, Finland; Catalog No. 8NT1), human proBNP (HyTest, Turku, Finland; Catalog No. 8PRO8), and glycosylated human proBNP ((HyTest, Turku, Finland; Catalog No. 8GOB2) were tested using a traditional two-step immunoassay format, as described below.

The two-step assay format occurs in a single reaction vessel. The two-step assay format comprises the steps of adding the sample, adding antibody coated magnetic microparticles, binding analyte, washing magnetic microparticle-analyte complexes, adding labeled conjugate antibody, binding of labeled conjugate to magnetic microparticle-analyte complexes, washing the resulting magnetic microparticle-analyte-labeled conjugate antibody complexes, and reading signal generated by the complexed label remaining in the reaction vessel.

Each sample dilution was dispensed in the amount of 100 μL into the individual reaction vessels. At the same time, 0.10% EDAC-coated magnetic microparticles (50 μL) (Polymer Science, Monticello, Ind.) coated with anti-human NT proBNP/anti-human proBNP monoclonal antibodies 15F11 (HyTest, Turku, Finland; Catalog No. 4TN1) or 15C4 (HyTest, Turku, Finland; Catalog No. 4TN1) were dispensed in the amount of 50 μL into the same reaction vessel. The reaction vessel was then vortexed to mix the sample and magnetic microparticles. Each reaction mixture was incubated for 18 minutes at 37° C.

During this incubation, any human NT-proBNP, human proBNP, or glycosylated human proBNP in the sample was capable of being captured by the anti-human NT-proBNP/anti-human proBNP monoclonal antibody coated onto the magnetic microparticles.

Upon completion of the 4 minute incubation, the magnetic microparticle human NT-proBNP/human proBNP antibody complexes were magnetically captured, and immobilized into a pellet on the side of the reaction vessel. The immobilized magnetic microparticle-human proBNP/human proBNP complex pellet was then washed by alternately aspirating the liquid from the vessel, and then adding assay kit wash buffer (ARCHITECT® wash buffer, available from Abbott Laboratories, Abbott Park, Ill.) into the reaction vessel (1 mL wash buffer, repeated 4 times). This process removes any unbound human NT-proBNP, human proBNP, and glycosylated human proBNP from reaction mixture. The magnetically captured microparticle-human NT-proBNP/human proBNP antibody complexes formed during the 4 minute incubation remain in the reaction vessel.

During a second incubation of 4 minutes, anti-human NT-proBNP/anti-human proBNP labeled monoclonal antibody 7B5 (HyTest, Turku, Finland; Catalog No. 4TN1) or 15C4 (HyTest, Turku, Finland; Catalog No. 4TN1) or 15F11 (HyTest, Turku, Finland; Catalog No. 4TN1) or 16F3 (HyTest, Turku, Finland; Catalog No. 4TN1) or 13G12 (HyTest, Turku, Finland; Catalog No. 4TN1) or 18H5 (HyTest, Turku, Finland; Catalog No. 4TN1) or 24E11 (HyTest, Turku, Finland; Catalog No. 4TN1) or 29D12 (HyTest, Turku, Finland; Catalog No. 4TN1) were each dispensed in the amount of 50 μL into individual reaction vessels containing only the 15F11 (HyTest, Turku, Finland; Catalog No. 4TN1) coated magnetic microparticles—human NT-proBNP/human proBNP complexes. This reaction mixture was then vortexed to disperse the microparticle pellet.

Also, during a second incubation of 4 minutes, anti-human NT-proBNP/human anti-proBNP labeled monoclonal antibody antibody 7B5 (HyTest, Turku, Finland; Catalog No. 4TN1) or 15C4 (HyTest, Turku, Finland; Catalog No. 4TN1) or 15F11 (HyTest, Turku, Finland; Catalog No. 4TN1) or 16F3 (HyTest, Turku, Finland; Catalog No. 4TN1) or 13G12 (HyTest, Turku, Finland; Catalog No. 4TN1) or 18H5 (HyTest, Turku, Finland; Catalog No. 4TN1) or 24E11 (HyTest, Turku, Finland; Catalog No. 4TN1) or 29D12 (HyTest, Turku, Finland; Catalog No. 4TN1) were each dispensed in the amount of 50 μL into individual reactions vessel containing only the 15C4 (HyTest, Turku, Finland; Catalog No. 4TN1) coated magnetic microparticle-human NT-proBNP/human proBNP complexes. This reaction mixture was then vortexed to disperse the microparticle pellet.

The magnetic microparticle-human NT-proBNP/human proBNP complexes were incubated with the labeled antibodies in buffer for 4 minutes at 37° C. During this incubation, the anti-human NT-proBNP/human anti-proBNP acridinium labeled antibodies also bind to the human NT-proBNP or human proBNP that was captured by the magnetic microparticles. This formed a microparticle-human NT proBNP or human proBNP-labeled antibody complex.

Upon completion of the 4 minute incubation, the magnetic microparticle-human NT-proBNP/human proBNP-labeled antibody complexes were again magnetically captured into a pellet. The recaptured pellet was then repeatedly washed with buffer (1 mL), repeated 4 times. The magnetically captured microparticle-human NT proBNP or human proBNP-labeled antibody complex pellet was then released.

The acridinium label (Abbott Laboratories, Abbott Park, Ill.) was then triggered to emit light. This was accomplished by adding a low pH (pH 1) buffer containing H2O2 (1.32%) (List 6E23-65, Abbott Laboratories, Abbott Park, Ill.) was dispensed (100 mL) to the microparticle complexes and vortexing. This step released the labeled anti-human NT-proBNP/anti-human proBNP acridinium (Abbott Laboratories, Abbott Park, Ill.) monoclonal antibody (Abbott Laboratories, Abbott Park, Ill.) that had been bound to human NT-proBNP or human proBNP captured by the microparticles.

The magnetic microparticles were then magnetically captured leaving the released acridinium labeled NT-proBNP or proBNP antibodies in the reaction mixture solution. This was followed by addition (300 mL) of a pH 13 buffer (List 6C55-60, Abbott Laboratories, Abbott Park, Ill.) which “triggers” light, relative light units (RLU), production from the acridinium released into the solution. The amount of light that was generated was used to determine the quantity of human NT-human proBNP, human proBNP, and glycosylated human proBNP detected in the sample (See, Table 1).

TABLE 1

| | | | Analyte rlus | | |
|---|---|---|---|---|---|
| Microparticle | Conjugate | | Human | Human | Human |
| Mab | Mab | pM | NT-proBNP | gly. proBNP | proBNP |
| 15F11 | 7B5 | 0.0 | 615 | 615 | 615 |
| | | 43.3 | 615 | 528 | 589 |
| | | 86.6 | 549 | 505 | 529 |
| | | 288.7 | 593 | 525 | 556 |
| | | 577.4 | 534 | 600 | 612 |
| 15F11 | 15C4 | 0.0 | 1345 | 1345 | 1345 |
| | | 43.3 | 21153 | 1263 | 1490 |
| | | 86.6 | 57226 | 2009 | 1544 |
| | | 288.7 | 312467 | 1577 | 2190 |
| | | 577.4 | 661509 | 1452 | 3358 |
| 15F11 | 15F11 | 0.0 | 912 | 912 | 912 |
| | | 43.3 | 904 | 899 | 857 |
| | | 86.6 | 929 | 886 | 963 |
| | | 288.7 | 977 | 925 | 949 |
| | | 577.4 | 1011 | 953 | 897 |
| 15F11 | 16F3 | 0.0 | 640 | 640 | 640 |
| | | 43.3 | 630 | 586 | 624 |
| | | 86.6 | 592 | 587 | 621 |
| | | 288.7 | 696 | 643 | 720 |
| | | 577.4 | 631 | 635 | 914 |
| 15F11 | 13G12 | 0.0 | 841 | 841 | 841 |
| | | 43.3 | 777 | 798 | 800 |
| | | 86.6 | 761 | 767 | 802 |
| | | 288.7 | 820 | 831 | 920 |
| | | 577.4 | 805 | 778 | 1076 |
| 15F11 | 18H5 | 0.0 | 626 | 626 | 626 |
| | | 43.3 | 621 | 618 | 628 |
| | | 86.6 | 636 | 737 | 664 |
| | | 288.7 | 708 | 663 | 949 |
| | | 577.4 | 664 | 754 | 1445 |
| 15F11 | 24E11 | 0.0 | 886 | 886 | 886 |
| | | 43.3 | 7381 | 863 | 904 |
| | | 86.6 | 20757 | 918 | 938 |
| | | 288.7 | 124174 | 906 | 1091 |
| | | 577.4 | 356904 | 892 | 1244 |
| 15F11 | 29D12 | 0.0 | 912 | 912 | 912 |
| | | 43.3 | 1986 | 2325 | 1569 |
| | | 86.6 | 3535 | 4474 | 2206 |
| | | 288.7 | 14573 | 16406 | 6145 |
| | | 577.4 | 33877 | 38714 | 11395 |

TABLE 2

| | | | Analyte rlus | | |
|---|---|---|---|---|---|
| Micropartic | Conjugate | | Human | Human | Human |
| Mab | Mab | pM | NT-proBNP | gly. proBNP | proBNP |
| 15C4 | 7B5 | 0.0 | 590 | 590 | 590 |
| | | 43.3 | 750 | 606 | 686 |
| | | 86.6 | 719 | 581 | 641 |
| | | 288.7 | 1178 | 672 | 607 |
| | | 577.4 | 2182 | 800 | 726 |
| 15C4 | 15C4 | 0.0 | 1941 | 1941 | 1941 |
| | | 43.3 | 1975 | 1805 | 2175 |
| | | 86.6 | 1995 | 1877 | 1824 |
| | | 288.7 | 2057 | 1862 | 1884 |
| | | 577.4 | 2078 | 1793 | 1955 |
| 15C4 | 15F11 | 0.0 | 847 | 847 | 847 |
| | | 43.3 | 883 | 1011 | 851 |
| | | 86.6 | 998 | 835 | 1035 |
| | | 288.7 | 1135 | 930 | 945 |
| | | 577.4 | 1372 | 859 | 917 |
| 15C4 | 16F3 | 0.0 | 1036 | 1036 | 1036 |
| | | 43.3 | 11382 | 691 | 1987 |
| | | 86.6 | 26463 | 861 | 4059 |
| | | 288.7 | 140366 | 827 | 13737 |
| | | 577.4 | 319526 | 1092 | 34681 |
| 15C4 | 13G12 | 0.0 | 892 | 892 | 892 |
| | | 43.3 | 10281 | 905 | 2217 |
| | | 86.6 | 25550 | 912 | 4076 |
| | | 288.7 | 131068 | 967 | 16707 |
| | | 577.4 | 308579 | 1242 | 37162 |
| 15C4 | 18H5 | 0.0 | 704 | 704 | 704 |
| | | 43.3 | 18028 | 756 | 3384 |
| | | 86.6 | 39142 | 783 | 6590 |
| | | 288.7 | 161543 | 1228 | ND |
| | | 577.4 | 336945 | 1582 | 46534 |
| 15C4 | 24E11 | 0.0 | 821 | 821 | 821 |
| | | 43.3 | 786 | 781 | 718 |
| | | 86.6 | 699 | 751 | 747 |
| | | 288.7 | 814 | 725 | 834 |
| | | 577.4 | 850 | 770 | 735 |
| 15C4 | 29D12 | 0.0 | 892 | 892 | 892 |
| | | 43.3 | 8867 | 877 | 2167 |
| | | 86.6 | 19329 | 1017 | 3834 |
| | | 288.7 | 82751 | 1222 | 12082 |
| | | 577.4 | 179728 | 1416 | 25910 |

TABLE 3

| | | |
|---|---|---|
| | | % Cross-reactivity |
| Magnetic | Acridinium | Human NT-proBNP |

| Micropartic | Conjugate | | Human | Human |
|---|---|---|---|---|
| Mab | Mab | pM | gly. proBNP | proBNP |
| 15C4 | 16F3 | 43.3 | −3.3 | 9.2 |
| | | 86.6 | −0.7 | 11.9 |
| | | 288.7 | −0.2 | 9.1 |
| | | 577.4 | 0.0 | 10.6 |
| 15C4 | 13G12 | 43.3 | 0.1 | 14.1 |
| | | 86.6 | 0.1 | 12.9 |
| | | 288.7 | 0.1 | 12.1 |
| | | 577.4 | 0.1 | 11.8 |
| 15C4 | 18H5 | 43.3 | 0.3 | 15.5 |
| | | 86.6 | 0.2 | 15.3 |
| | | 288.7 | 0.3 | nd |
| | | 577.4 | 0.3 | 13.6 |
| 15C4 | 29D12 | 43.3 | −0.2 | 16.0 |
| | | 86.6 | 0.7 | 16.0 |
| | | 288.7 | 0.4 | 13.7 |
| | | 577.4 | 0.3 | 14.0 |
| 15F11 | 15C4 | 43.3 | −0.4 | 0.7 |
| | | 86.6 | 1.2 | 0.4 |
| | | 288.7 | 0.1 | 0.3 |
| | | 577.4 | 0.0 | 0.3 |
| 15F11 | 24E11 | 43.3 | −0.4 | 0.3 |
| | | 86.6 | 0.2 | 0.3 |
| | | 288.7 | 0.0 | 0.2 |
| | | 577.4 | 0.0 | 0.1 |
| 15F11 | 29D12 | 43.3 | 131.6 | 61.2 |
| | | 86.6 | 135.8 | 49.3 |
| | | 288.7 | 113.4 | 38.3 |
| | | 577.4 | 114.7 | 31.8 |

*Note, in this Table 3, some combinations were omitted because of a lack of significant reactivity.

### DISCUSSION OF THE RESULTS

A two step sandwich immunoassay was used to quantitate human NT-proBNP, human glycosylated proBNP and human proBNP with HyTest anti-human NT-proBNP/anti human proBNP monoclonal antibodies.

Two anti-human NT-proBNP/anti-human proBNP monoclonal antibodies were separately coated onto magnetic microparticles and used for human NT-proBNP, human glycosylated proBNP, and human proBNP immunoassay capture. Eight labeled anti-human NT-proBNP/anti-human proBNP monoclonal antibodies were used for human NT-proBNP, human glycosylated proBNP and human proBNP immunoassay detection. A total of sixteen combinations of HyTest anti-human NT-proBNP/anti-human proBNP monoclonal antibodies were tested (See, Table 4).

TABLE 4

| Magnetic | | Labeled | |
|---|---|---|---|
| Microparticles | Epitope | Conjugate | Epitope |
| Coating Mab | a.a. | Mab | a.a. |
| 15C4 | 63-71 | 7B5 | 13-24 |
| 15C4 | 63-71 | 15C4 | 63-71 |
| 15C4 | 63-71 | 15F11 | 13-24 |
| 15C4 | 63-71 | 16F3 | 13-20 |
| 15C4 | 63-71 | 13G12 | 13-20 |
| 15C4 | 63-71 | 18H5 | 13-20 |
| 15C4 | 63-71 | 24E11 | 67-76 |
| 15C4 | 63-71 | 29D12 | 5-12 |
| 15F11 | 13-24 | 7B5 | 13-24 |
| 15F11 | 13-24 | 15C4 | 63-71 |
| 15F11 | 13-24 | 15F11 | 13-24 |
| 15F11 | 13-24 | 16F3 | 13-20 |
| 15F11 | 13-24 | 13G12 | 13-20 |
| 15F11 | 13-24 | 18H5 | 13-20 |
| 15F11 | 13-24 | 24E11 | 67-76 |
| 15F11 | 13-24 | 29D12 | 5-12 |

Seven of the tested combinations detected human NT-proBNP (See, Table 5). Four of these combinations (15C4 anti-human NT-proBNP/human anti-proBNP monoclonal antibody coated magnetic microparticles used with labeled antibodies 16F3, 13G12, 18H5, and 29D12 (anti-human NT-proBNP/human anti-proBNP monoclonal antibodies)) also cross-reacted between 9% and 15% with equimolar concentrations of human proBNP (See, Table 3). This result was not unexpected since human proBNP shares all the epitopes found on human NT-proBNP.

15F11 anti-human NT-proBNP/human anti-proBNP monoclonal antibody coated magnetic microparticles tested with 29D12 labeled, anti-human NT-proBNP/human anti-proBNP monoclonal antibody, reacted relatively weakly (33877 rlus, 577 pM) with human NT-proBNP compared to (38714 rlus, 577 pM) with human glycosylated proBNP, and (11395 rlus, 577 pM) with human pro-BNP (See, Table 1). This result was not unexpected considering human NT-proBNP also shares epitopes with human glycosylated proBNP and human proBNP.

Two anti-human NT-proBNP/anti-human proBNP monoclonal combinations using 15F11 anti-human NT-proBNP/human anti-proBNP monoclonal antibody coated onto magnetic microparticles, with either 15C4 or 24E11 anti-human NT-proBNP/human anti-proBNP monoclonal labeled antibodies, readily detected human NT-proBNP but cross reacted with human proBNP less than about 1%. (See, Table 3). This finding was surprising and unexpected. Specifically, considering that human NT-proBNP shares the same epitopes with human glycosylated proBNP and human proBNP, one skilled in the art would expect that human proBNP would also be detected.

The anti-human NT/proBNP monoclonal antibodies combinations which readily detect human NT-proBNP, but do not cross-react with human glycosylated proBNP (e.g., less than about <1%) or human proBNP (e.g., less than about <1%), are based on human NT-proBNP amino acid residues 13-34 (15F11) magnetic microparticle capture and human NT proBNP amino acids residues 63-71 (15C4) or amino acid residues 67-76 (24E11) for detection.

The anti-human NT/proBNP monoclonal antibodies combinations that used a reverse amino acid capture (13-20), and detection (63-71), resulted in significant cross-reactity (˜10%) with human proBNP.

TABLE 5

| | Epitope | Con- | Epitope | Human | Human | |
|---|---|---|---|---|---|---|
| Microparticle | Amino | jugate | Amino | NT- | gly. | Human |
| Mab | A. | Mab | A. | proBNP | proBNP | proBNP |
| 15C4 | 63-71 | 16F3 | 13-20 | + | − | + |
| 15C4 | 63-71 | 13G12 | 13-20 | + | − | + |
| 15C4 | 63-71 | 18H5 | 13-20 | + | − | + |
| 15C4 | 63-71 | 29D12 | 5-12 | + | − | + |
| 15F11 | 13-24 | 15C4 | 63-71 | + | − | − |
| 15F11 | 13-24 | 24E11 | 67-76 | + | − | − |
| 15F11 | 13-24 | 29D12 | 5-12 | + | + | + |

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.
The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An immunoassay for quantifying the amount of human NT-pro B-type natriuretic peptide (“human NT-proBNP”) in a test sample being tested for or suspected of containing human NT-proBNP, the immunoassay having reduced cross-reactivity with any human pro B-type natriuretic peptide (“human proBNP”) present in the test sample and comprising the steps of:
(a) contacting at least one capture antibody that binds to human NT-proBNP and that has been immobilized onto a solid phase to produce an immobilized antibody with said test sample to form a first mixture comprising an at least one capture antibody-human NT-proBNP complex, wherein said capture antibody comprises antibody 15F11;
(b) contacting said first mixture comprising the at least one capture antibody-human NT-proBNP complex with at least one detection antibody that binds to human NT-proBNP and that has been conjugated to a detectable label to form a second mixture comprising at least one capture antibody-human NT-proBNP-at least one detection antibody complex, wherein the detection antibody comprises antibody 24E11 or 15C4; and
(c) determining the amount of the at least one capture antibody-human NT-proBNP-at least one detection antibody complex formed in step (b) by detecting the detectable label as a measure of the amount of human NT-proBNP contained in the test sample,
wherein the at least one capture antibody and the at least one detection antibody, when used together, exhibit a cross-reactivity of less than about 1.0% with any human proBNP present in the test sample.

2. The immunoassay of claim 1, wherein the at least one detection antibody is 24E11.

3. The immunoassay of claim 1, wherein the at least one detection antibody is 15C4.

4. An immunoassay for quantifying the amount of human NT-pro B-type natriuretic peptide (“human NT-proBNP”) in a test sample being tested for or suspected of containing human NT-proBNP, the immunoassay having reduced cross-reactivity with any human pro B-type natriuretic peptide (“human proBNP”) present in the test sample and comprising the steps of:
(a) contacting said test sample with at least one detection antibody that binds to human NT-proBNP and that has been conjugated to a detectable label to form a first mixture comprising an at least one human NT-proBNP-detection antibody complex, wherein the detection antibody comprises antibody 24E11 or 15C4;
(b) contacting said first mixture comprising said at least one human NT-proBNP-detection antibody complex with at least one capture antibody that binds to human NT-proBNP and that has been immobilized on to a solid phase to produce an immobilized antibody to form a second mixture comprising an at least one capture antibody-human NT-proBNP-at least one detection antibody complex, wherein said at least one capture antibody comprises antibody 15F11; and
(c) determining the amount of the at least one capture antibody-human NT-proBNP-at least one detection antibody complex formed in step (b) by detecting the detectable label as a measure of the amount of human NT-proBNP contained in the test sample,
wherein the at least one capture antibody and the at least one detection antibody, when used together, exhibit a cross-reactivity of less than about 1.0% with any human proBNP present in the test sample.

5. The immunoassay of claim 4, wherein the at least one detection antibody is 24E11.

6. The immunoassay of claim 4, wherein the at least one detection antibody is 15C4.

7. An immunoassay for quantifying the amount of human NT-pro B-type natriuretic peptide (“human NT-proBNP”) in a test sample being tested for or suspected of containing human NT-proBNP, the immunoassay having reduced cross-reactivity with any human pro B-type natriuretic peptide (“human proBNP”) present in the test sample and comprising the steps of:
(a) contacting a test sample with at least one capture antibody that binds to human NT-proBNP and that has been immobilized onto a solid phase to produce an immobilized antibody and with at least one detection antibody that binds to human NT-proBNP and that has been conjugated to a detectable label to form an at least one capture antibody-human NT-proBNP-at least one detection antibody complex, wherein the at least one capture antibody comprises antibody 15F11 and the at least one detection antibody is antibody 24E11 or 15C4; and
(b) determining the amount of the at least one capture antibody-human NT-proBNP-at least one detection antibody complex formed in step (a) by detecting the detectable label as a measure of the amount of human NT-proBNP contained in the test sample, wherein the at least one capture antibody and the at least one second antibody conjugated to the detectable label, when used together, exhibit a cross-reactivity of less than about 1.0% with any human proBNP present in the test sample.

8. The immunoassay of claim 7, wherein the at least one detection antibody is 24E11.

9. The immunoassay of claim 7, wherein the at least one detection antibody is 15C4.

10. An immunodiagnostic reagent comprising at least one capture antibody and at least one detection antibody specific for human NT-pro B-type natriuretic peptide (“human NT-proBNP”), and that, when used together, exhibit a cross-reactivity of less than about 1.0% with any human pro B-type natriuretic peptide (“human proBNP”).

11. The immunodiagnostic reagent of claim 10, wherein the capture antibody is antibody 15F11 and the detection antibody is antibody 24E11 or 15C4.

12. A kit for the detection of human NT-pro B-type natriuretic peptide (“human NT-proBNP”) in a test sample, said kit comprising:
(a) instructions for conducting the assay of the test sample; and
(b) an immunodiagnostic reagent that comprises at least one capture antibody and at least one detection antibody specific for human NT-pro B-type natriuretic peptide (“human NT-proBNP”), and that, when used together, exhibit a cross-reactivity of less than about 1.0% with any human pro B-type natriuretic peptide (“human proBNP”).

13. The kit of claim 12, wherein capture antibody is antibody 15F11 and the detection antibody is antibody 24E11 or 15C4.


SEQUENCE LISTING (4 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 76 aa, Homo sapiens
MDPQTAPSRALLLLLFLHLAFLGGRSHPLGSPGSASDLETSGLQEQRNHLQGKLSELQVEQTSLEPLQESPRPTGV

SEQ ID NO: 4 - protein, 32 aa, Homo sapiens
SPKMVQGSGCFGRKMDRISSSSGLGCKVLRRH